(19) United States
(12) Patent Application Publication
(10) Pub. No.: US 2016/0363578 A1
(43) Pub. Date: Dec. 15, 2016

(54) APPARATUS FOR DETERMINING MEASUREMENT VARIABLES AT MEMBRANES

(71) Applicant: CYTOCENTRICS BIOSCIENCE GMBH, Rostock (DE)

(72) Inventors: Thomas Knott, Rostock (DE); Michael Huber, Horbranz (AT); Peter Van Stiphout, KH Helmond (NL)

(21) Appl. No.: 15/121,370

(22) PCT Filed: Feb. 18, 2015
(86) PCT No.: PCT/EP2015/053416

§ 371 Date: Aug. 24, 2016

(30) Foreign Application Priority Data
Feb. 24, 2014 (DE) 10 2014 203 280.6

(51) Int. Cl. G01N 33/487; G01N 27/30; G01N 27/327; G01N 27/416

(52) U.S. Cl. CPC G01N 33/48728; G01N 27/4167; G01N 27/4166; G01N 27/302; G01N 27/304; G01N 27/327

(57) ABSTRACT

An apparatus for determining preferably Biologically Relevant Measurement Variables at membranes has at least one first plate-like or film-like substrate, which is made from at least one first material and has continuous openings or pores or can be perforated, and also has a second plate-like or film-like substrate which is made from at least one second material, to which measuring elements are assigned or on which measuring elements are situated. Cells or similar structures may be at least temporarily applied or permanently cultivated on the first substrate. According to the invention, the first substrate and the second substrate are assigned to one another at least during the measuring operation for determining the corresponding measurement variables, specifically preferably on flat sides which are opposite one another. In particular, this assignment is a connection of the two substrates, preferably a joining-together.

The invention relates to an apparatus for determining preferably biologically relevant measurement variables at membranes, a substrate compound, which is suitable for such a determination, as well as a method which can be performed with the assistance of the named apparatus.

The determination of measurement variables of electrochemical gradients, potentials, resistances or charge transfers which occur at membranes, in particular at cell membranes or similar structures, has already been known for a long time. We refer for example to the disclosures of WO 02/10747 A2 for a technical background, in particular to the introductory explanations of this publication.

In this connection, measurement methods and measurement devices are also known with which (normally thin) flat substrates which are fitted with continuous openings, i.e. are perforated, are implemented. We refer to an example of US 2006/0194255 A1 as a disclosure of the current state of the art.

In any case, such flat substrates used for Patch Clamp with Gigaseal are generally limited to use on cells in suspension, i.e. whole cell or perforated Patch Clamp is only possible on cells in suspension which are applied to fresh substrates. If a contact between cells and substrate is already present, or cells are cultivated on the substrate, the seal resistance typically lies in the range of 100 mega-ohms (Megaseal), and gigaohms (Gigaseals) can no longer be reached.

For most cells, the viability is dramatically limited by individualization and/or suspension. Cells in suspension—individual or not—can achieve a maximum viability of a few minutes up to perhaps a few hours. Exceptions to this are only those cells which are naturally in suspension (such as blood cells).

Adherent cells, i.e. cells which adhere to other cells or other structures, or cells in cell groups additionally exhibit biological phenomena (such as stimulus conduction or induction) which cannot be observed in a single suspended cell. A method is therefore desired that can be implemented for random cells or tissue without waiving the intracellular measurement parameters captured by the Patch Clamp. Also desirable is to capture other or further measurement parameters, such as for example impedance, energy metabolism and extracellular potential.

To date, the implementation of substrates for measuring impedance, capturing energy metabolism and measuring extracellular potentials was frequently performed by direct placement of the measurement elements and electrodes on the upper surface of the corresponding substrate—there where the cells to be examined also adhere and, if required, provide demands to the substrate which counter the characteristics of the measurement elements on the substrate.

The task of this invention is to produce a reversible measurement situation for numerous cells (individual, coherent or in a combination), whether suspended, adherent or in a tissue composite.

The task of this invention is also to structure the measurement elements, including the (measurement) electrodes in such a manner that these are connected to the cells and tissues to be measured via the perforation or patch pipette, for example to make a cell-friendly surface possible, or for example, to obtain easier technical access to the measurement elements.

Finally, the task of this invention is to equip the scope of the apparatus with substrates in order to capture numerous measurement parameters. Targeted measurement parameters are intracellular potentials or currents, such as for example, those captured by the Gigaseal Patch Clamp, impedance, energy metabolism, pH to measurement of extracellular potentials or for extracellular stimulation.

These tasks are at least partially resolved by the apparatus with the characteristics of claim 1, the composite with the characteristics of claim 18 and the method with the characteristics of claim 20. Preferred embodiments are described in the respective dependent claims. The text for all claims is herewith expressly made part of the contents of this description.

The apparatus according to the invention for the determination of preferably biologically-relevant measurement variables—such as electrochemical gradient, potential, resistance, charge and shifting or altering thereof—on membranes which exhibit or produce membrane proteins such as for example, ion channels, in particular on cell membranes or similar structures, is characterized in that the apparatus exhibits
at least a first plate-like or film-like substrate which consists of at least one first material and has continuous openings or pores or can be perforated, and on which cells can be applied individually or in a tissue composite, or similar structures can be applied, at least temporarily, or permanently cultivated,
at least a second plate-like or film-like substrate which consists of at least a second material to which the measurement elements are assigned or on which the measurement elements are situated,
whereby the first substrate and the second substrate are assigned to one another at least during the measurement process for determining the corresponding measurement variables, preferably on flat sides which are opposite one another, in particular connected with one another or connectable with one another, preferably joined together or capable of being joined together.

Preferably the distance of both substrates before and/or during the measurement operation is adjustable, preferably variably adjustable.

The term “substrate” should be understood comprehensively according to the invention, i.e. it concerns a material according to a type of carrier, a base plate or similar, on which the structures, components and similar are necessary for the performance of the Patch Clamp measurements are formed or provided. In the present case, this relates to a flat substrate in the form of a plate or film. This should mean that such substrates generally exhibit a larger width or length, in particular a significantly greater width or length in comparison to the thickness (or height) of such a flat substrate. The width and length thereby define both flat sides of the substrate. Thereby, the thickness of a film-type substrate, which can also be structured to be flexible, is generally less than the thickness of a plate-type substrate.

“Microfluidic components” or “microfluidic structures” are components, structures, parts and similar, which allow storing, moving, mixing, separating or other handling of liquid media on small and smallest areas. Liquid media can thereby contain dissolved gases. As explained above, the membranes or cells to be examined in the corresponding Patch Clamp measurements are generally handled in the form of suspensions in suitable solvents, and thereby, among other things, applied to the corresponding Patch Clamp opening, or transported away therefrom. Such suspensions are guided through the named microfluidic components and structures. The corresponding specialty area is known to the expert in the field under the term microfluidics.

“Membranes” in the sense of the invention should be understood as all separation layers or also layers with other functions which can be examined using the Patch Clamp technique. This can relate to artificial membranes which, for example, can be realized in the form of double lipid layers. The so-called bio-membranes, which are preferably examined according to the invention, generally refer to separation layers between various areas within a living cell (intracellular) or also between the inside of a cell and the space outside of a cell. Thereby, according to the invention, individual membrane fragments, complete membranes or also complete cells are examined. Cell composites are also comprised in the invention.

According to the invention, the assignment of both substrates can be performed in such a manner that the second substrate is situated on the same side of the first substrate as the cells on whose membrane the measurement is to be performed. Accordingly, in such embodiments, the cells with their membranes are situated between the first and second substrate.

In further embodiments, the assignment between the two substrates is realized in such a manner that the second substrate is situated on the side opposite (away from) those of the cells on the first substrate.

Generally, the assignment of both substrates, in particular their connection or joining them together, can be performed mechanically, pneumatically, magnetically, electrostatically, chemically or capillary forces. The corresponding forces ensure that both substrates are brought close to one another or are brought together and, if necessary, can be held there.

The apparatus according to the invention advantageously exhibits additional means for adjusting and/or for maintaining a distance from both substrates in their assignment, whereby these means are preferably mating depressions, recesses, projections and similar that fit together in the first substrate and/or in the second substrate.

The orientation of both substrates to one another, i.e. the adjustment, can be performed optically, mechanically, electromechanically, electrically, magnetically or acoustically, whereby the means implemented in these cases must ensure that both substrates are oriented and remain in the correct geometric order, if applicable, with the correct distance from one another.

In a further development, at least one channel is formed with the assignment of both substrates which can be contacted fluidically and/or electrically, whereby, the distance to both substrates is adjustable, in particular hydraulically or pneumatically, preferably with the assistance of the channel.

In the apparatus according to the invention, the total thickness and total height of the first substrate or the second substrate lies between 0.1 μm and 1 mm, preferably between 0.1 μm and 100 μm, in particular between 0.1 μm and 20 μm, in particular between 0.1 μm and 10 μm.

In the apparatus according to the invention, the total thickness or total height of the first substrate is preferably smaller than the total thickness or total height of the second substrate, whereby the total thickness or total height of the first substrate is preferably 0.2× to 0.8×, in particular 0.3× to 0.6× the total thickness or total height of the second substrate.

According to the invention, it is further preferred if the diameter or the largest cross-section measurement of the opening(s) or pore(s) in the first substrate lies between 0.01 μm and 1 mm, preferably between 0.01 μm and 100 μm, in particular between 0.01 μm and 20 μm, in particular between 0.1 μm and 20 μm.

The surface measurements of the first and second substrates used according to the invention i.e. the measurements of their flat sides lie in the range of the measurement variables of common cell culture containers. Here, surface measurements of these containers which are available in very many sizes and forms, lie between 1 mm² and 1,000 cm². Surface measurements of a common microtiter plate are preferable, for example with side lengths of 8 cm to 12 cm. Common measurements are also those of common cell culture containers between 10 cm² and 250 cm², for example, containers with surfaces of 25 cm², 75 cm² and 175 cm². Naturally, any smaller substrates can also be implemented which, for example, also cover only a portion of a named container.

In a further development, the first material the first substrate is preferably a dielectric material, in particular silicon, silicon dioxide, silicon nitride, aluminum oxide, glass and/or a plastic.

Alternatively, the first material of the first substrate can preferably be a metal, a film coated with metal and/or a conductive plastic, whereby the total thickness or total height of the first substrate is preferably between 1 μm and 20 μm.

In a further development of the apparatus, the first substrate preferably consists of at least two layers of different materials, whereby at least one of the cells or the similar structures of the neighboring layer are preferably made of at least a first material.

In this connection it is emphasized that, in particular so-called functional coatings can be provided on the first substrate, which can assist in influencing the immobilization of cells or similar structures on the corresponding substrate. Such functional coatings are preferably provided around the openings or pores of the first substrate.

Functional coatings serve, for example, to select the cells to be contacted from a suspension which contains the relevant target cells in addition to other cells or structures.

This isolated aspect of the invention is, for example, described in WO 02/03058 A2, the disclosure of which is made part of this description, at least in this regard.

Preferably in the invention, at least one measurement chamber is assigned to the first substrate, in which, for example, the cells with the membrane to be examined during the measuring procedure is fixed or immobilized on the corresponding opening or pore in the first substrate. Thereby the contact to these measurement chambers in the first substrate is preferably performed on the cells on the opposite side of the first substrate via the liquid bridges which are produced by the openings and pores which are present in the first substrate. If necessary, the moisture on the side opposite the cells (underside of the first substrate) can be advantageously controlled and limited if necessary via the hydrophobia or hydrophillia of the materials of the first substrate on the side opposite the cells.

The measurement elements which are assigned to the second substrate or are situated thereon in this invention, can preferably be electrodes and/or biosensors according to the invention whereby the electrodes are preferably formed in a planar or especially in the form of three-dimensional bodies such as needles, cylinders, cubes, tetrahedrals, prisms, mushroom shape and similar.

The named electrodes can preferably convey the contacts of liquid bridges, the measurement of oxygen or other dissolved gases, the measurement of the pH value or the measurement of extracellular potentials, and the extracellular stimulation with voltage and/or current pulses.

The measurement elements can thereby be introduced or applied to the second substrate with chemical or physical methods and, if possibly, if advantageous, also in the first substrate. Alternatively, or additionally, imprinting processes can be implemented, such as so-called micro-imprinting.

Concerning the electrodes used as measuring elements, it is advantageous if at least one electrode is provided on the first substrate, whereby preferably at least one electrode is assigned to each of both sides of this first substrate. If the flat sides of the substrate are arranged horizontal in the functional state of the apparatus according to the invention, then the corresponding electrodes are situated above and/or below the first substrate.

In the cases in which the first substrate (at least partially) consists of a conductive material, the substrate material itself can form the corresponding electrode, whereby in the case of available measurement chambers, preferably at least one electrode is provided for each measurement chamber.

The apparatus according the invention preferably exhibits at least one channel on the second substrate as a measurement element, preferably numerous channels, similar to a Patch Clamp pipette, which primarily protrude away from the second substrate, and through the inside of which, at least during the measurement procedure, a fluidic, in particular microfluidic contact, is possible.

In the presence of these channels (Patch Clamp pipettes), a connection the first and second substrates is possible in a manner that the channels/pipettes on the second substrate can be brought to the openings/pores of the first substrate and, if necessary, can be guided through these openings/pores. In this manner, the piercing of a perforation present on the first substrate can be performed. The described method makes it possible for the channels/pipettes to the reach cells on the first substrate and contact these. Thereby, the actual measurement procedure in such embodiments is made possible in a particularly effective manner.

For the named embodiments, it is advantageous if an electrode is assigned to each channel/each pipette which is situated either within the channel/pipette or in the microfluidic or even macrofluidic inflow to these channels/pipettes.

Advantageous in the invention altogether, and in particular in the last-mentioned embodiments, is the presence of up to 4,000 channels/pipettes or measurement contacts per square millimeter (mm²) on the second substrate.

The channels similar to a type of Patch Clamp pipette on the second substrate thereby exhibit is in particular a length or height between 0.1 μm and 1 mm, an outer diameter or an outer largest cross-section measurement between 0.1 μm and 1 mm, and a wall thickness between 0.01 μm and 500 μm.

The apparatus according to the invention is preferably characterized in that in the second material of the second substrate is, at least partially, a semiconductor or in particular a dielectric material, which is especially suitable for forming a so-called gigaseal, whereby the second material is preferably silicon, silicon dioxide, silicon nitride, aluminum oxide and/or glass.

The second substrate also preferably consists of at least two layers of different materials.

In a further development the apparatus is preferably structured in such a manner that only the channels provided on the second substrate are, like a Patch Clamp pipette, at least partially consist of a semiconductor or in particular at least partially of a dielectric material, whereby preferably only the area of the ends of the channels protruding away from the second substrate, i.e. the tips of the so-called Patch Clamp pipettes, consist of this material.

The invention further comprises a composite of two plate-type or film-type substrates for use in determining preferably biologically relevant measurement variables at membranes which exhibit or produce membrane proteins such as, for example, ion channels, in particular on membranes of cells or similar structures, whereby a first substrate has at least one first material with continuous openings or pores or can be perforated, and on this substrate, cells, individually or in a tissue composite, or similar structures can be applied at least temporarily or permanently cultured, and a second substrate consisting of at least one second material,
on which measurement elements are assigned or on which the measurement elements are situated, preferably on the opposing flat sides, in particular connected reversibly or irreversibly, preferably joined together.

The distance of both substrates in this composite before and/or during the measurement procedure is preferably adjustable, preferably variably adjustable.

In relation to further preferred characteristics of the composites according to the invention with at least a first substrate and at least a second substrate, reference is expressly made to the prior embodiments in connection with the apparatus according to the invention. The characteristics named there can, as long as they are referring to the substrates, also be subject of the claimed composite.

Finally, the invention also comprises a method for determining preferably biologically relevant measurement variables—such as electrochemical gradients, potential, resistance, charge and the shifting or altering thereof—on membranes which exhibit or produce membrane proteins such as, for example, ion channels, in particular on cell membranes or similar structures, characterized in that two plate-type or film-type substrates are provided, whereby a first substrate is comprised of at least a first material which exhibits continuous openings or pores or can be perforated, and on which substrate cells, individual or in a tissue composite, or similar structures can be applied at least temporarily or permanently cultivated, and whereby a second substrate consisting of at least one second material is assigned to measurement elements or measurement elements are situated on this substrate, and whereby the first substrate and the second substrate for determining the measurement variables in the provided measurement procedure, preferably on the other flat side, which are either already in mutually assigned, or already assigned, especially connected state or so organized just before the measurement procedure, especially connected to one another.

With this method, preferably the distance of both substrates before and/or during the measurement procedure are preferably adjustable, preferably variably adjustable.

In regards to further preferred characteristics of the method according to the invention, reference is made to the previous embodiments in connection with the apparatus according to the invention. The characteristics named there can also be object of the claimed method.

According to the invention, the apparatus preferably exhibits at least one plate-type or foil-type substrate consisting of a first material (either conducting or non-conducting), which is in particular not gigaseal capable. The substrate is very thin and easy to perforate, or provided with continuous openings. It is preferably thinner or less thick than the pipette height of the second substrate (which can also be configured as conducting or non-conducting). The second substrate—plate-type or foil-type—contains measuring elements and is preferably gigaseal-capable. Preferably there are one or more pipette tips thereon it which are contacted microfluidically. If necessary, only the pipettes are gigaseal-capable, if applicable, also only the tips of the pipettes.

The second substrate is connected with the first substrate for the measurement procedure. Connecting both substrates can thereby be performed in an irreversible manner, for example by gluing, or reversibly using releasable connections. During connecting, the pipettes of the second substrate preferably enter into the opening of the first substrate, or perforate the first substrate.

The openings or pores in the first substrate are continuous according to the invention, i.e. they run from one side of the substrate (if applicable, the ‘top’ in the functional state) to the other side of the substrate (if applicable, the ‘bottom’). These openings/pores preferably exhibit a circular cross-sectional surface, whereby however other cross-section forms (square, rectangular, oval or similar) are also generally possible. The cross-section surfaces of the openings/pores can be continuous over the course of the opening/pore, or taper to one side of the substrate.

The pipettes on the second substrate protrude up to 1,000 μm over the substrate base surface of this second substrate, preferably however only a few 10 μm, preferably however only a few μm.

It is understood that not only openings or pipettes of the same type (in regards to size, form and similar) can be provided in a substrate but rather, various openings and pipettes of various types simultaneously.

The openings and also the pipettes can be installed in any manner into the corresponding materials of which the substrates are formed. Preferably the openings and pipettes are produced chemically or physically, for example through an etching process with chemicals.

For measurement, cells or tissue are applied to the first substrate and, if necessary, cultivated there. The measurement is performed immediately after applying the cells/tissue, or any time during the cells/tissue in culture.

Significant points in the invention are also three “enabling factors.”

1) With the invention it is possible that both substrates are brought closer together for the measurement. Thereby a separate channel is produced which is fluidically contacted. Preferably, the pipettes in the second substrate penetrate through the perforations of the first substrate or through the existing openings in the first substrate to the cells situated on the first substrate and form the measurement contact (preferably with Gigaseal). After the measurement, both substrates can then be separated again and the cells on the first substrate can be further cultivated, if applicable.

2) Fluid running out through the openings of the first substrate during handling of the first substrate (as long as the first and second substrate are not connected) can be prevented through suitable measures, such as for example with a hydrophobic underside on the first substrate or, for example, with a transport dish filled with liquid for the first substrate.

Pipettes used on the second substrate are “stripped” before being used again, that is, separated electrically and/or fluidically. Unused pipettes, i.e. those pipettes which did not reach a cell in the previous measurement procedure and were used for a measurement process, can be selectively used again without a cleaning step. After cleaning, all pipettes, used or unused, which were not mechanically destroyed during use can be used again.

Prior to connecting substrate one and substrate two, a selected sub-volume of the pipettes can be stripped using a selection/control of microfluidic contacts (targeted), so that the corresponding pipettes do not perform a measurement process and therefore remain unused.

3) Intracellular perfusion is also possible through microfluidic contacts of the pipettes.

For the sake of completeness, reference is made that the substrate or the carrier/take-up must not necessarily completely consist of the corresponding material (first material and/or second material). This can also be a coating consisting of the corresponding materials which are then, for example, applied to a base consisting of a completely different material, for example a plastic material.

In addition to research on electrically active tissues, the invention can also be implemented, for example, in cancer diagnostics, for example in order to examine the sensitivity of cancer cells removed from the human body (ex-vivo) to various chemotherapeutic agents in vitro.

Special aspects of the invention can also be demonstrated with the following description:

An array of measurement chambers on a perforated or perforatable first substrate independent of a material with order and diameter (0.01-1,000 μm) of the openings and thickness (0.1-1,000 μm) which are adapted to the respective measurement variables on which first substrate individual or suspended cells or tissues or natural or artificial vesicles can be applied through gravitation and/or fluid flows or on which cell cultures or tissue cultures can be grown or applied. The first substrate is thereby provided with a coating and/or a mechanical, chemical or biological surface modification, so that the growth or the adherence of the cell culture is stimulated or inhibited, or the oxygen measurement, pH measurement or extracellular measurement is improved or made easier, for example, by coating with poly-lysine or other proteins. Measurement electrodes or measurement devices for oxygen measurement, pH measurement or extracellular measurement or stimulation are either applied on the substrate or below the substrate or introduced into the substrate.

According to the invention, a second substrate, particularly one made of silicon, silicon oxide or glass is brought so close to the first substrate that the pipettes on the second substrate penetrate into the first substrate and possibly penetrate it so that Teraseals or at least Gigaseals are possible on the cells on the first substrate through the pipettes on the second substrate. In any case, so close that the measurement elements on the second substrate can perform measurements on the cells on the first substrate.

The cells on the first substrate can alternatively be situated on the same side as the second substrate, and thus make it easier for the second substrate to approach the cells.

The resulting sandwich structure from the first and second substrate is preferably only maintained for the measurement procedure. The second substrate with the pipettes or with its own measurement elements consists of a carrier consisting of, for example, of silicon, silicon oxide or glass, and preferably contains microfluidically-contacted pipettes (0.1-1,000 μm thickness, 0.1-1,000 μm high) consisting of of silicon oxide, silicon nitride, aluminum oxide or other dielectric materials which make a gigaseal possible. The ratio of pipettes to measurement chambers is n:m. The first substrate with the measurement chambers and the second substrate are directly connected via a connecting element.

Each individual or numerous electrode(s) are arranged above and each individual or numerous electrode(s) below the first substrate in such a manner that at least one electrode is situated in each measurement chamber which is suitable for capturing extracellular potentials such as spikes, total or field potentials, to measure impedance and intracellular voltage and current terminals.

In addition, one or numerous oxygen-sensitive electrode(s) and one or numerous pH-sensitive electrodes(s) are arranged above or below the first substrate or on both sides of the substrate in such a manner that at least one oxygen-sensitive or one pH-sensitive electrode is located in each measurement chamber which are suitable to capture, for example, energy metabolism.

In addition, one or numerous channels are arranged on the upper and lower side of the first substrate and the second substrate which allows the introduction of cells and the change of media, culture media, pore formers for perforated patch and pharmacologically, biologically or chemically-active substances.

The named and further characteristics as well as advantages of the invention result from the following description of figures in connection with the sub-claims. Thereby, the individual characteristics of the invention can be realized alone or in combination with one another. The embodiments described in following merely serve to explain and provide better understanding of the invention, and are not in any way to be understood as restrictive.

The drawings show

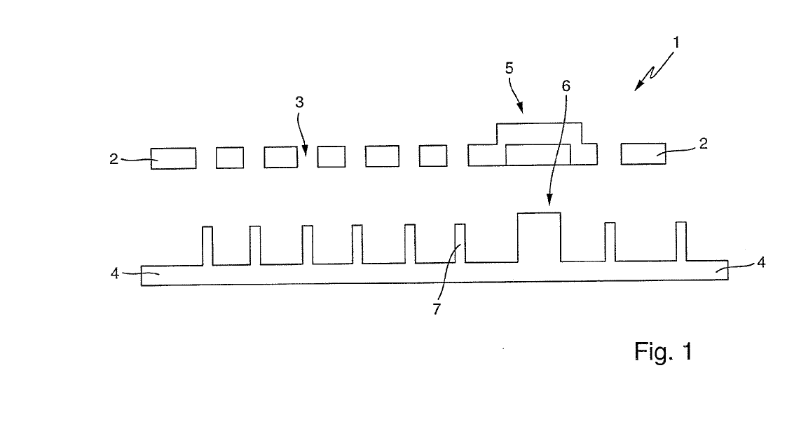

FIG. 1 a first embodiment of the apparatus according to the invention in a schematic cut view

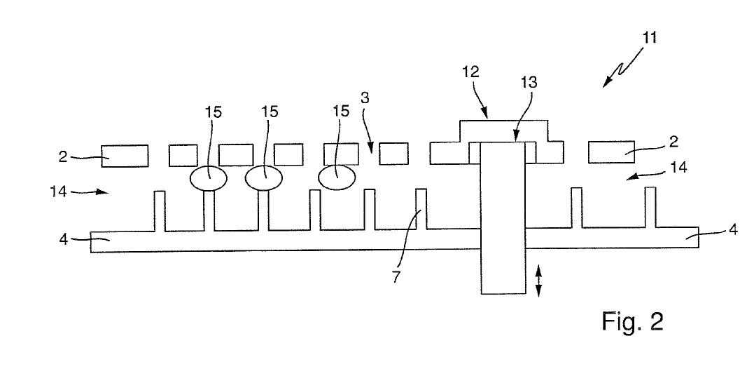

FIG. 2 a second embodiment of the apparatus according to the invention in a schematic cut view,

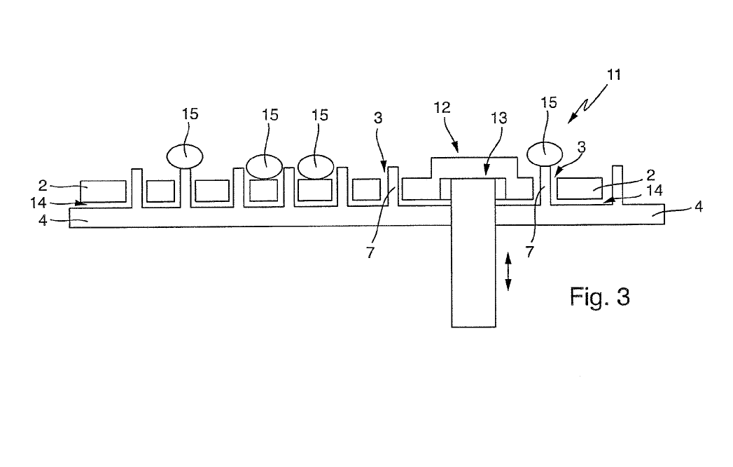

FIG. 3 the second embodiment of the apparatus according to the invention in a further application and

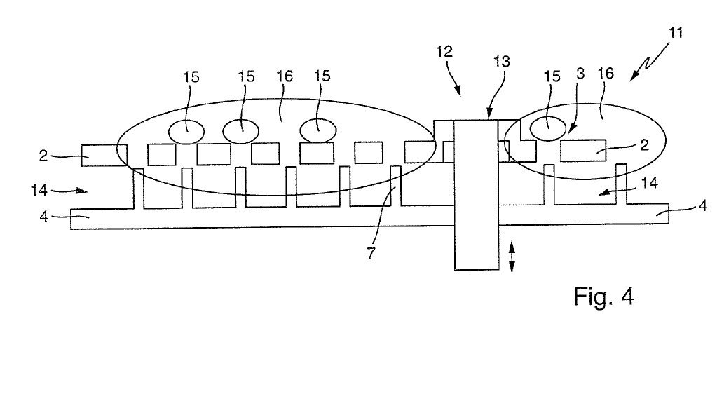

FIG. 4 the second embodiment of the apparatus according to the invention in a further application.

According to FIG. 1, the apparatus 1 comprises two substrates 2 and 4, which are formed plate-type or film-type. The first substrate 2 exhibits numerous, in particular a multiplicity of openings or pores 3 which are guided in a vertical direction through the substrate 2. The presented case in FIG. 1 thereby depicts pores 3 with essentially circular cross-sectional areas.

FIG. 1 further depicts the second substrate 4 in which numerous, preferably a multiplicity of channels 7 like a patch-clamp pipette protrude in the vertical direction from the base surface of the substrate 4. The channels 7 that cannot be individually depicted in FIG. 1 are hollow on the inside, so that for example a fluid exchange and/or an electrical contact through these channels is possible.

Further, according to FIG. 1, the apparatus 1 on the substrates 2 and 4, means 5, 6 are provided, which are provided for orientation or adjustment of the substrates 2 and 4 towards one another. In the case presented, in FIG. 1 the means 5 formed on the substrate are a cavity or recess, which can work together (at least partially) with a fitting means 6 in the form of a protrusion on the substrate 4 in such a manner that the position of both substrates 2 and 4 are defined towards one another. In this manner, both substrates 2 and 4 are arranged towards one another, in particular their connection, for example in the form of joining together in a simple manner.

The orientation of both substrates 2 and 4 towards one another with the means 5 and 6, thereby is preferably set in a manner that the pipette-type channels 7 are applied on substrate 4 in the correct position to interact with the pores on substrate 2.

Liquid inlet lines, for example to the pipettes 7 on the substrate 4 as well as an electrical contact, in particular via corresponding electrodes, are not presented in FIG. 1 for reasons of clarity. Also not presented is that substances can be rinsed through all accesses to the cells which have effects on the membranes, ion channels or cells. These substances can also be gases. The respective partial pressure of the various gases can thereby be adjusted or measured in a targeted manner, for example using partial oxygen pressure, anaerobic metabolism can be differentiated from aerobic metabolism, or achieved stimulating specific receptors using nitrogen monoxide (NO).

The apparatus 1 according to FIG. 1 can be used in various manners according to the invention. It is thereby possible, for example, to arrange cells whose membranes are to be examined between the (first) substrate 2 and the (second) substrate 4. It is also possible, for example, to arrange corresponding cells on the (flat) side of the (first) substrate 2 which is on the other side of the (second) substrate 4. In the presentation chosen in FIG. 1, the cells lie on the upper (upper flat side) of the (first) substrate 2.

Both of the last described possibilities are explained in greater detail in the following figures.

The embodiment depicted in FIG. 2 of the apparatus 11 according to the invention varies from the embodiment depicted in FIG. 1 primarily only in that instead of the means 5 and 6 depicted in FIG. 1, other means 12 and 13 are provided to orient and separate both substrates from one another.

The means 12 and 13 refer to a recess 12 in substrate 2 which is comparable to a recess 5 in FIG. 1. This recess can interact with a vertically movable protrusion 13 on substrate 4, so that the distance of both substrates 2 and 4 from one another can be variably adjusted in the apparatus 11.

As soon as both substrates 2 and 4 so close to one another that the means 13 on the substrate 4 interact with the means 12 on substrate 2, a channel is formed between the two substrates which allows a fluidic and/or electrical contact between both substrates 2 and 4. The width (as shown in FIG. 2, the height) of this channel 14 can be varied with the assistance of the variably adjustable means 12 and 13, and set as desired. This adjustment possibility is symbolically presented in FIG. 2 with the double arrow at means 13.

Further, FIG. 2 depicts the possibility to arrange and immobilize cells 15 between both substrates 2 and 4 in the apparatus 11 depicted there. Thereby internal or external membranes of these cells 15 can be examined in such an arrangement with a corresponding Patch Clamp measurement. This possibility is depicted in FIG. 2 in the left and middle cells 15. There, the corresponding cells are both immobilized on the respective tips of both pipettes 7 and ready for a Patch Clamp measurement.

A further application of the apparatus 11 according to the invention is depicted in FIG. 3. As in FIG. 2, the apparatus 11 differentiate from apparatus 1 as per FIG. 1 primarily only through the means for orienting and maintaining a distance of both substrates 2 and 4.

According to FIG. 3, both substrates 2 and 4 are arranged towards one another in such a manner that they are nearly completely connected. The (still) resulting narrow distance of both substrates 2 and 4 from one another can be defined and adjusted with the assistance of the means 12 and 13. A channel remains here between the (first) substrate 2 and the (second) substrate 4 over which the fluidic or electrical contact is possible.

In the embodiment according to FIG. 3, the pipette-type channels 7 formed on the substrate 4 penetrate pores/openings 3 assigned thereto, whereby the tips of these pipette-type channels, as depicted in FIG. 3, can preferably protrude over the upper flat side of the substrate 2.

In an embodiment as in FIG. 3, substrates 2 can also be implemented in which the openings and pores 3 in their original state are not yet or not yet completely formed. In such cases, this relates to an embodiment in which the substrate 2 is perforatable. Then the actual opening/pore 3 cannot be (completely) formed until the associated pipette-type channel 7 penetrates the perforatable substrate 2, here in the vertical direction.

In the embodiment according to FIG. 3, cells 15 are arranged or immobilized on the upper flat side of the substrate 2, i.e. on the side opposite the substrate 4. Thereby, according to FIG. 3 the three cells 15, which are situated on the respective tips of the channels 7 are ready for a Patch

Clamp measurement. In the presentation in FIG. 3, these are the cells 15 which are arranged on the outer left and both cells on the right.

FIG. 4 finally depicts a further application, in which the apparatus 11 according to the invention can be implemented. In this embodiment, a comparable distance is set between the (first) substrate 2 and the (second) substrate 4 with the help of means 12 and 13. The resulting channel 14 is therefore comparatively wide (or high in the presentation according to FIG. 4).

Despite this, a measurement according the invention can also be performed is such cases via both fluid bridges, which are presented in FIG. 4 as oval areas 16. In this manner, a fluidic contact can be provided between the liquid which is situated in the pipette-type channels 7, and the cells/cell membranes with these areas 16. These contacts are performed through substrate 2 without bringing these channels 7, into direct contact with the cells or their membranes, as for example in the embodiment according to FIG. 3.

The electrical contact takes place in the embodiment depicted in FIG. 4 exactly as in the embodiments according to FIGS. 1 to 3 using electrodes which are provided on substrate 4 and/or in the channels 7, and which are situated above the substrate 2 with the help of electrodes.

1. Apparatus for determining preferably biologically-relevant measurement variables—such as electrochemical gradient, potential, resistance, charge and shifting or alteration thereof—at membranes which exhibit or produce membrane proteins such as, for example, ion channels, in particular on membranes of cells or similar structures, characterized in that the apparatus
Has at least a first plate-type or film-type substrate which consists of at least a first material which has continuous openings or pores or which can be perforated, and on which the cells can be applied individually or in a tissue composite, or similar structures at least temporarily, or can be permanently cultivated,
at least a second plate-type or film-type substrate consisting of at least a second material, which measuring elements are assigned to or on which measurement elements are situated,
whereby the first substrate and the second substrate are preferably arranged on flat sides opposite one another, at least during the measurement procedure for the determination of the corresponding measurement variables, facing one another, in particular connected with one another, or preferably joined together or capable of being joined together.

2. Apparatus according to claim 1, characterized in that the distance of both substrates before and/or during the measurement procedure is adjustable, preferably variably adjustable.

3. Apparatus according to claim 1 or claim 2, characterized in that the additional means for orientation and/or to maintain a distance to both substrates in their arrangement is provided whereby these means are preferably recesses, hollows, protrusions and similar that fit together in the first substrate and/or in the second substrate.

4. Apparatus according to claim 1, characterized in that at least one channel is formed with the assignment of both substrates which can be fluidically and/or electrically contacted, whereby the distance of both substrates can preferably be adjusted with the channel, in particular hydraulically or pneumatically adjustable.

5. Apparatus according to claim 1, characterized in that the total thickness or total height of the first substrate or the second substrate is between 0.1 μm and 1 mm, preferably between 0.1 μm and 100 μm, in particular between 0.1 μm and 20 μm, in particular between 0.1 μm and 10 μm.

6. Apparatus according to claim 1, characterized in that the total thickness or total height of the first substrate is smaller than the total thickness or total height of the second substrate, whereby the total thickness or total height of the first substrate is preferably 0.2× to 0.8×, in particular 0.3× to 0.6× the total thickness or total height of the second substrate.

7. Apparatus according to claim 1, characterized in that the diameter or the largest cross-section measurement of the opening or pore in the first substrate is between 0.01 μm and 1 mm, preferably between 0.01 μm and 100 μm, in particular between 0.01 μm and 20 μm, in particular between 0.1 μm and 20 μm.

8. Apparatus according to claim 1, characterized in that the first material of the first substrate is a dielectric material, in particular silicon, silicon dioxide, silicon nitride, aluminum dioxide, glass and/or a plastic.

9. Apparatus according to claim 1, characterized in that the first material of the first substrate is a metal, a film coated with metal and/or a conductive plastic, whereby the total thickness or total height of the first substrate is preferably between 1 μm and 20 μm.

10. Apparatus according to claim 8 or claim 9, characterized in that the first substrate is comprised of at least two layers of different materials, whereby at least one of the layers facing towards the cells or the similar structures consists of at least a first material.

11. Apparatus according to claim 1, characterized in that the measurement elements assigned to the second substrate or situated thereon are electrodes and/or biosensors, whereby the electrodes are preferably planar, or especially formed as three-dimensional bodies such as needles, cylinders, pyramids, cubes, tetrahedrons, prisms, mushroom-shaped or similar.

12. Apparatus according to claim 1, characterized in that through electrodes, the contact of liquid bridges, the measurement of oxygen or other dissolved gases, the measurement of pH values or the measurement of extracellular potentials are determined and the extracellular stimulation with voltage and/or current pulses.

13. Apparatus according to claim 1, characterized in that on the second substrate at least one channel is provided as a measurement element, preferably a multiplicity of channels, formed similar to a Patch Clamp pipette, which protrudes primarily vertically from the second substrate, and through which, at least during the measurement procedure, a fluidic, in particular microfluidic contact is possible.

14. Apparatus according to claim 13, characterized in that the channels like a Patch Clamp pipette on the second substrate exhibit a length or height between 0.1 μm and 1 mm, an outer diameter or an outer largest cross-sectional measurement between 0.1 μm and 1 mm, and a wall thickness between 0.01 μm and 500 μm.

15. Apparatus according to claim 1, characterized in that the second material in the second substrate is at least partially a semiconductor or in particular a dielectric material which is in particular suitable for forming a so-called gigaseal, whereby the second material is preferably silicon, silicon dioxide, silicon nitride, aluminum oxide and/or glass.

16. Apparatus according to claim 15, characterized in that the second substrate consist of at least two layers made of different materials.

17. Apparatus according to claim 15 or claim 16, characterized in that only the channels on the second substrate like a Patch Clamp pipette are at least partially made of a semiconductor or in particular at least partially made of a dielectric material, whereby preferably only the area of the ends of the channels protruding from the channels, i.e. the tips of the so-called Patch Clamp pipettes, are made of this material.

18. Compound of two plate-type or film-type substrates for use in determining preferably biologically-relevant measurement variables at membranes which exhibit or produce membrane proteins, such as for example ion channels, in particular on membranes of cells or similar structures, whereby a first substrate exhibits continuous openings or pores consisting of at least a first material, or can be perforated, and cells individually or in tissue compound or similar structure, can be temporarily applied to or permanently cultured and a second substrate consisting of at least a second material, which the measurement elements are assigned to or situated on the measurement elements, preferably on flat sides opposite one another, arranged towards one another, in particular reversibly or irreversibly connected with one another, preferably joined together.

19. Compound according to claim 18, characterized such that the distance of both substrates before and/or during the measurement procedure is adjustable, preferably variably adjustable.

20. Method for determining preferably biologically-relevant measurement variables—such as electrochemical gradient, potential, resistance, charge and shifting or alteration thereof—on membranes which exhibit or produce membrane proteins such as for example ion channels, in particular on cell membranes or other structures, characterized in that the two plate-type or film-type substrates are provided, whereby a first substrate consisting of at least a first material exhibits continuous openings or pores or which can be perforated, and cells, individually or in tissue composites, or similar structures can be applied to this substrate at least temporarily or be permanently cultivated, and whereby a second substrate consisting of at least a second material is assigned measurement elements or on which measurement elements are situated, and whereby the first substrate and the second substrate specified for determining measurement variables in a measurement process are preferably on opposite flat sides, either already in an arranged state opposite one another, in particular in the connected state, or are fitted to one another directly before the measurement process, in particular joined together.

21. Method according to claim 20, characterized in that the distance of both substrates before and/or during the measurement process is adjustable, preferably variably adjustable.
